(19) United States
(12) Patent Application Publication
(10) Pub. No.: US 2008/0125977 A1
(43) Pub. Date: May 29, 2008

(54) USE OF QUANTUM SYSTEM IDENTIFICATION AND QUANTUM CONTROL TECHNIQUES FOR MEDICAL DIAGNOSTIC AND THERAPEUTIC PURPOSES

(75) Inventors: Patrick A. ANQUETIL, Brookline, MA (US); Ian W. HUNTER, Lincoln, MA (US); Serge LAFONTAINE, Lincoln, MA (US); Shyamal SOMAROO, West New York, NJ (US); Ching-Hua Tseng, Belmont, MA (US)

Correspondence Address: WILMERHALE/DC, 1875 PENNSYLVANIA AVE., NW, WASHINGTON, DC, 20004, US

(73) Assignee: Aretais, Inc., Lincoln, MA (US)

(21) Appl. No.: 11/927,622

(22) Filed: Oct. 29, 2007

Related U.S. Application Data
(60) Provisional application No. 60/855,072, filed on Oct. 27, 2006.

(51) Int. Cl. G01N 33/48; G01J 3/44

(52) U.S. Cl. 702/19; 356/301

(57) ABSTRACT

Quantum simulation methods are used to encode the quantum response of a molecular system so as to improve the sensitivity for detection of a target material, while rejecting background. The perturbation and response information may be used to discover the system function of a quantum system, or more generally, of a complex system, such as a physiological system. The approach may be applied to medical non-invasive, real-time, continuous molecular detection and quantification techniques through coherent Raman spectroscopy to enable a significantly more attractive course of therapy than existing protocols.

### CROSS-REFERENCE TO RELATED APPLICATIONS

This application claims the priority of U.S. Provisional Application No. 60/855,072, filed Oct. 27, 2006 and entitled “Use of Quantum System Identification and Quantum Control Techniques for Medical Diagnostic and Therapeutic Purposes,” the whole of which is hereby incorporated by reference herein.

### GENERAL AREA OF TECHNOLOGY

The invention relates to adaptive control techniques to study molecules.

### BACKGROUND

Detection, identification, and quantification of substances are often limited by the signal strength or the available signal to noise ratio. This restriction is particularly pronounced in medical diagnostics. Although non-invasive detection and measurement of substances in biological tissue, such as the human body, is possible using optical techniques, such techniques have been difficult and time consuming to implement in practice. One of the biggest challenges to practical implementation involves increasing the sensitivity to the signal and enhancing background rejection.

Presently, most non-invasive detection methods are either not sensitive enough to low levels of target molecules in a large background or cannot be calibrated to provide an absolute concentration value. Even for instances where an in vitro sample may be available, it would advantageous to have better sensitivity detection schemes to allow for real-time analysis. A need exists for more sensitive optical detection methods, in particular to quantify a known target molecular species.

Coherent Raman spectroscopy is one optical method that may be particularly useful for identifying target molecular species. Coherent Raman is a non-linear optical spectroscopic technique that returns an optically coherent response from a sample. The coherency induced in the target molecule can increase signal by several orders of magnitude compared to incoherent Raman spectroscopy, thereby reducing the time required to perform measurements. Generally, coherent Raman involves multiple pumps and probe lasers and can therefore be seen as a driven process. Unfortunately, along with the coherent resonant response is a large confounding non-resonant background signal. To manage, reduce, or eliminate this background, various forms of coherent Raman techniques have been developed. For strongly scattering media, however, many of these techniques are considered impractical because of the scrambling of spectral phases and polarization.

Quantum control has been recently explored by some as a way to increase further the detected target molecular signal and to discriminate against other molecular contributions. Quantum control may be applied to enhance optical spectroscopic methods, such as Raman spectroscopy and its derivatives. Some of these methods include, but are not limited to: femtosecond adaptive spectroscopic techniques for coherent anti-Stokes Raman spectroscopy (FAST CARS) and its coherent Stokes Raman spectroscopy (CSRS) analogue; hybrid CARS spectroscopy, and time resolved CARS and CSRS detection. Scully, for instance, has disclosed an approach whereby the steps to achieve a desired quantum state of a target molecule is optimized in an adaptive fashion, so as to increase the detected target molecular signal and to discriminate against contribution to signal from other molecular species. [U.S. Pat. No. 6,795,777; Scully, M. O., Kattawar, G. W., Lucht, R. P., Opatmy, T., Pilloff, H., Rebane, A., Sokolov, A. V., and Zubairy, M. S., “FAST CARS: Engineering a laser spectroscopic technique for rapid identification of bacterial spores.” PNAS 99, 10994 (2002).]

Judson and Rabitz have used laser pulses as “optical reagents.” They have used quantum dynamical discrimination whereby a feedback loop with an algorithm optimizing a cost function adjusts a series of control pulses. [Rabitz, H., “Shaped laser pulses as reagents.” Science 299, 525 (2003); Judson, R. S., Rabitz, H., Phys. Rev. Lett. 68, 1500 (1992); U.S. Pub. No. 2004/0128081, 2005/0230239, 2005/0240311, and 2006/0271305]. In this feedback loop, a pulse sequence may eventually be discovered that optimally prepares the quantum system in some predefined way; either for controlling a reaction path or for improving the “product” yield caused by the illumination. The laser pulse sequence itself, which is found by this adaptive feedback scheme, can also serve as a representative fingerprint of the molecule. Silberberg uses a similar adaptive feedback loop to empirically formulate an ultrashort laser pulse sequence, without explicitly requiring theoretical and predictive knowledge of the solution. [U.S. Pat. Nos. 6,327,068 and 6,621,613, U.S. Pub. No. 2002/0044328]. Silberberg also describes coherent Raman methods using unitary pulses for coherent optical control. [U.S. Pub. 2004/0145735].

Each of these references proposes ways to attain a quantum state that will give the strongest signal from a target molecule. In many instances, however, the maximized signal from the target may be still too weak to detect readily. The signal from background, for instance, may be so large that it obscures the signal of the target molecule. A need therefore exists to more effectively extract and measure the signal of a target molecule when the signal from the target may be difficult observe.

### SUMMARY

A method and system is provided to identify one or more molecular species in an environment that ordinarily interferes with the detection of the molecular species. This method is particularly well suited for analytical tests of biological materials, either ex vivo or in vivo, to determine the biochemical or hematological characteristics of the material, or to measure the concentration in such material of proteins, hormones, carbohydrates, lipids, drugs, toxins, gases, electrolytes, metabolites, etc. The method is particularly well suited for measuring physiological system properties such as the glucose-insulin response. In one or more embodiments of the identification method, quantum control is used to guide, through a sequence of quantum mechanical states, the evolution of a quantum mechanical system in a target molecule. In the process, information is encoded onto a target molecule's quantum system, which can be later “decoded” to help identify the presence of the target molecule.

Methods of manipulating the quantum states of a molecule or collection of molecules, informed by the detected response to a perturbation, are provided so as to optimize the detected quantum states. Under one aspect, we describe a quantum control method that includes exposing a sample containing a target having a quantum state to a first electromagnetic radiation to enforce a first Hamiltonian on said quantum state of said target, wherein said radiation encodes a pattern on said quantum state of said target; detecting a response from said sample; calculating an expected response of said target to said first electromagnetic radiation; and comparing said expected response of said target to said detected response from said sample to measure the presence of said target in said sample.

Some embodiments include one or more of the following features: finding an optimal pulse sequence with a feedback loop to maximize a signal response from said target, wherein the feedback loop comprises: detecting said signal response from said target after exposing said target to a second electromagnetic radiation to enforce a second Hamiltonian on said quantum state of said target; calculating a cost function; modifying said second electromagnetic radiation based on said cost function; and exposing said target to said modified second electromagnetic radiation. The first and second Hamiltonian, in some embodiments, may be time dependent or time independent. The second electromagnetic radiation may be modified according to an adaptive algorithm, such as a genetic, landscape algorithm, or an algorithm based on evolution strategies, evolutionary programming, simulated annealing, Gaussian adaptation, hill climbing, or swarm intelligence. Said first electromagnetic radiation may be encoded with a pseudo random binary perturbation pattern, a swept frequency perturbation pattern, a stochastic input drive perturbation pattern, a regular frequency or multiple frequency pattern, or other non-regular perturbation pattern. Said first electromagnetic radiation may be from a laser, NMR, microwave, ultrasound, or radiofrequency generator. The signal response may arise from Raman scattering.

Under another aspect, we describe a system identification method that includes perturbing a system having a system function so as to determine said system function of said system; encoding a signal on said system; decoding said signal from said system to identify a target material in said system.

Some embodiments include one or more of the following features: said system may be a quantum system, such as a biological molecule or therapeutic composition. The biological molecule may be at least one member selected from the group consisting of glucose, insulin, viral marker, immune marker, cardiovascular biomarker, inflammation biomarker, cholesterol, triglycerides, C-reactive protein, bilirubin, alkaline phosphatase, alanine aminotransferase, AST/GOT, TSH, creatine, creatinine, albumin, cerebral spinal fluid analyte, Tau protein, Alzheimer's biomarker, blood brain barrier transport biomarker, ocular aqueous humor analyte, plaque precursor, cancer antigen, toxicity biomarker, metabolic biomarker, transport biomarker, hemoglobin, diabetes biomarker, central nervous system biomarker, and urogenital biomarker.

Some embodiments provide systems and methods for encoding and decoding the quantum evolution of a system.

Some embodiments provide systems and methods for optically measuring molecules in a sample whereby molecular species are identified and/or quantified by coherent Raman spectroscopy with a feedback loop. Some embodiments provide systems and methods for minimally invasive or non-invasive medical diagnostics of molecules such as blood glucose, viral or immune markers, cardiovascular or inflammation biomarkers, cholesterol, triglycerides, C-reactive protein, bilirubin, alkaline phosphatase, alanine aminotransferase, AST/GOT, TSH, creatine, creatinine, albumin, cerebral spinal fluid analytes, Tau protein Alzheimer's biomarkers, blood brain barrier transport biomarkers, ocular aqueous humor analytes, plaque precursors, cancer antigens such as the prostate specific antigen (PSA), metabolism and toxicity biomarkers, transport biomarkers, hemoglobin, diabetes biomarkers, central nervous system biomarkers, and urogenital biomarkers.

Some embodiments allow for a high sensitivity and discrimination of spectroscopic signals from physiologically and medically relevant analytes relative to the background through the use of quantum control and system identification methods, particularly optical methods such as FAST CARS as well as time resolved CARS and time resolved CSRS, to allow for optimal quantum mechanical state preparation, manipulation, and detection.

### BRIEF DESCRIPTION OF THE DRAWINGS

FIG. 1 schematically illustrates a CARS system for manipulating quantum states of a quantum system.

FIG. 2 is a schematic view diagram for optimizing a perturbation to attain a user-defined objective quantum state or series of states.

FIG. 3 is a schematic view of a system for characterizing the system function of a system with a feedback loop.

FIG. 4 is a schematic view of a system for encoding and decoding signal on a and from a quantum system.

### DETAILED DESCRIPTION OF THE INVENTION

Quantum control is typically accomplished using pulses of electromagnetic radiation (e.g., radio or optical pulses) so as to enforce upon a target molecule a particular quantum state. We use quantum control to encode a pattern onto the quantum evolution of a molecular system, which can be used to help identify the molecular system against a large background of noise. The methodology and system, which are described herein to manipulate and guide the quantum evolution of a molecular system, rests on principles of quantum information processing, [see, e.g., Viola, L., Loyd, S., and Knill, E., “Universal control of decoupled quantum systems.” Phys. Rev. arXiv: quant-ph/9906094 v1, 1 (1999)], and system identification.

System identification is a sophisticated signal processing technique that can treat the target system on multiple levels of complexity. [Korenberg, M. J. and Hunter, I. W. The identification of nonlinear biological systems: Volterra kernel approaches. Annals of Biomedical Engineering, 1996, 24, 250-268; Lennart Ljung: System Identification—Theory For the User, 2nd ed, PTR Prentice Hall, Upper Saddle River, N.J., 1999; Dudovich, N., Oron, D., and Silberberg, Y., “Coherent Transient Enhancement of Optically Induced Resonant Transitions.” Phys. Rev. Lett. 88, 123004 (2002)]. System identification allows us to generate experimentally models, such as the nonlinear dynamic relation between insulin delivery and blood glucose level, as well as the molecular dynamic behavior of glucose. Although these models provide no direct physical insight into the underlying physiology of the pancreatic control system or quantum mechanism underlying the behavior of glucose, they allow one to predict the behavior of the system. Furthermore the models provide systems and methods to perturb the system in order to explore and understand the response of the system.

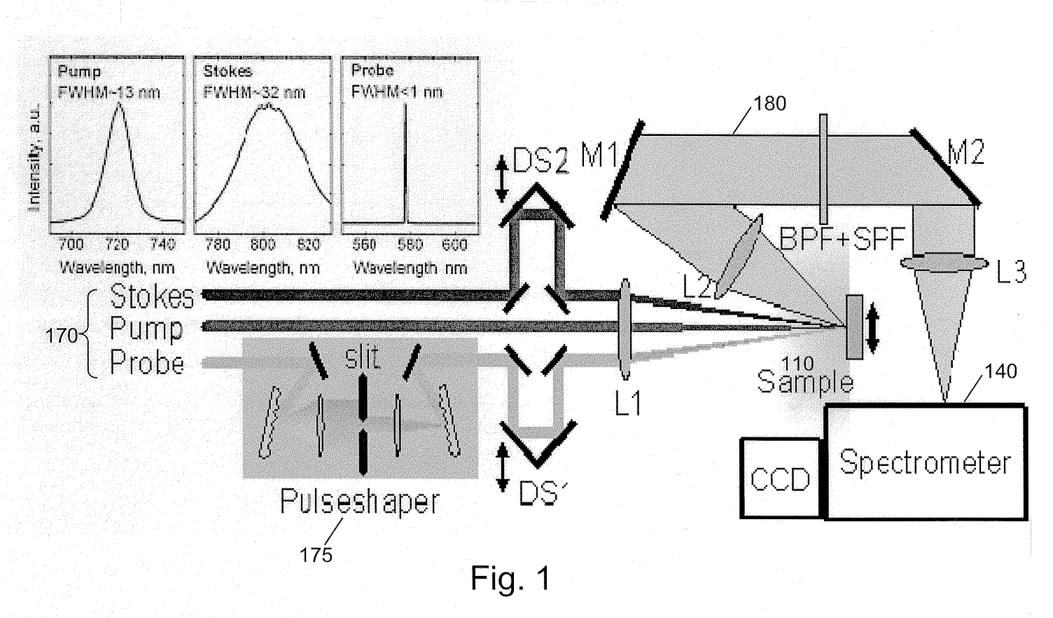

In one embodiment, we use coherent optical techniques to carry out the quantum state manipulation. A laser system for coherent Raman spectroscopy may be used for instance. FIG. 1 shows one embodiment of a suitable laser system. A Ti:Sapphire regenerative amplifier (not shown) (Legend, Coherent: 1 kHz rep. rate, ˜1 mJ/pulse) evenly pumps two optical parametric amplifiers (also not shown) (OPAs) (OPerA-VIS/UV and OPerA-SFG/UV, Coherent). The output of the first OPA (λ₁=712-742 nm, tunable; FWHM˜12 nm) and a small fraction of the amplifier output (λ₂=803 nm, FWHM˜32 nm) are used as pump and Stokes beams, respectively. The output of the second OPA is used as a probe beam (λ₃=578 nm) and sent through a pulse shaper 175, such as an adjustable slit, that modulates the bandwidth of the pulse.

The Stokes and probe pulses pass through respective delay stages (DS1, DS2), that adjust their relative timing appropriately, and then all three beams 170 are focused by a convex 2-inch lens L1 (with the focal length f=200 mm) onto a sample 110. The scattered light 180 is collected with a 2-inch achromatic lens (f=100 mm) and focused onto the entrance slit of a spectrometer (Chromex Spectrograph 250is) with a liquid nitrogen cooled charge-coupled device (CCD: Spec-10, Princeton Instruments) attached. The spectrometer 140 is in communication with a computer system (not shown) that digitally records and stores information about the scattered light 180. This information can then be used to manipulate molecules in the sample, as described in greater detail herein.

In the described embodiment of the laser system, the probe pulse is spectrally narrowed. To remedy in part the sacrifice of pulse energy lost by the slit, a thicker nonlinear crystals is used for the frequency conversion process that produces the probe pulse. The thicker nonlinear crystal results in a narrower probe spectrum to start with and therefore higher throughput of the pulse shaper. Alternatively, the second OPA can, in principle, be replaced with a second harmonic crystal, placed in the fundamental beam. Or one can also use other wavelengths simultaneously generated in the first OPA to simplify the setup.

Other systems to prepare and control the quantum mechanical state of a target molecule may be used, including but not limited to nuclear magnetic resonance (NMR), trapped ions, quantum dots, cavity quantum electrodynamics, optical pumping, bose-einstein condensates, and superconductors. Other alternative systems that are capable of controlling the quantum state of a target molecular species in accordance with the methods and system disclosed herein is exemplified in Scully, M. O., Zhu, S. Y., “Quantum Control of the Inevitable.” Science 281, 1973 (1998); Scully, M. O., Kattawar, G. W., Lucht, R. P., Opatmy, T., Pilloff, H., Rebane, A., Sokolov, A. V., and Zubairy, M. S., “FAST CARS: Engineering a laser spectroscopic technique for rapid identification of bacterial spores.” PNAS 99, 10994 (2002); Rabitz, H., “Shaped laser pulses as reagents.” Science 299, 525 (2003); Judson, R. S., Rabitz, H., Phys. Rev. Lett. 68, 1500 (1992); Dudovich, N., Oron, D., and Silberberg, Y., “Quantum Control of the Angular Momentum Distribution in Multiphoton Absorption Processes.” Phys. Rev. Lett. 92, 103003 (2004). Any other suitable method known to those skilled in the art to control the quantum state of a target may be used.

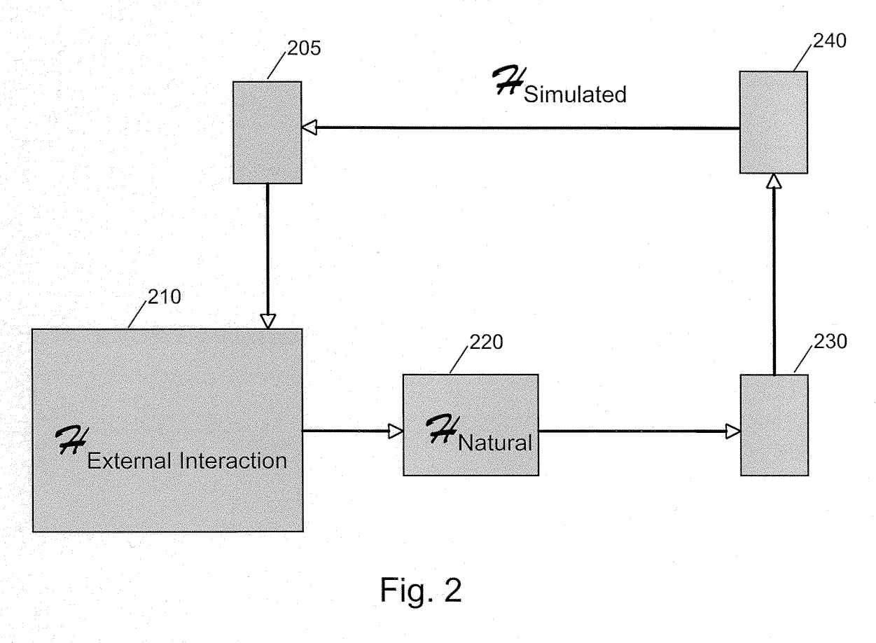

FIG. 2 schematically illustrates how an external Hamiltonian, such as that provided by an electromagnetic field, through interaction with the system manipulates and controls the quantum state of a molecule. An external interaction Hamiltonian 210, controlled by controller 205 operates on physical target 220 having a natural Hamiltonian, which ordinarily controls the quantum state behavior of the target, such as its molecular motion and intramolecular dynamics. The evolutionary response of target 220 to interaction Hamiltonian 210 in combination with the target's natural Hamiltonian may be observed by detector 230. For purposes of quantum control, data analyzer 240 compares the response of the target 220 to a user defined objective, and a cost function is calculated. The cost function is a fitness landscape, wherein a position in parameter space has an associated value or fitness that is maximized or minimized through movement in the parameter space. Since the space is often very large, strategies to optimize this cost function include iterative search algorithms. One common approach is to start with an initial random population, or points in the parameter space, calculate the result or cost for each member (or random subset), then “mutate” the parameter choices and select the most “fit” members to produce the next generation. Iteratively, this hopefully will yield a satisfying solution.

Subsequently, instructions are sent to controller 205 to adjust the interaction Hamiltonian 210, e.g., the parameters of the applied preparation and Stokes pump pulses. This process may be repeated until the cost function is minimized, i.e., the optimal pulse or pulse sequence (interaction Hamiltonian) is determined to achieve the desired objective. In the case of coherent Raman, for instance, the objective may be a maximally coherent vibrational state to achieve maximal Raman scattering.

Adaptive algorithms, such as genetic algorithms, evolution strategies, evolutionary programming, simulated annealing, Gaussian adaptation, hill climbing, and swarm intelligence, may be used to efficiently find the optimal pulse conditions to achieve the desired molecular quantum coherence. The control over the quantum state of a target may be achieved by adjusting any number of variables of the pulsed laser system, including, but not limited to, the number of pulses used for each perturbation, the type (e.g., frequency, shape, phase, amplitude), duration, and the timing of these pulses. To restrict the otherwise prohibitively vast number of variables of a laser pulse, one or more parameters of the laser pulse maybe fixed. A seeding of likely solutions can help the convergence of the search algorithm. Also, the inclusion of some non-fit members in the generating set will help maintain diversity of parameter choices and avoid premature convergence. The determined optimal pulse parameters is itself then reflective of the glucose vibrational level structure, and may thus be considered akin to a fingerprint of the molecule in that a different molecule would have a different optimal pulse sequence.

Alternatively, the maximum vibrational coherence of a molecular may be adiabatically driven, that is to be driven slowly enough so as to maintain the quantum system in an instantaneous eigenstate, by a laser with constant amplitude and swept frequency. This alternative (perhaps a noisy light field with slower time variation in offset frequency) may provide a way to use adiabatic laser fields instead of pulsed fields.

As a demonstration of quantum control, Somaroo et al. have shown that one quantum system can be simulated by another. [Quantum Simulations on a Quantum Computer. S. S. Somaroo, C. H. Tseng, T. F. Havel, and D. G. Cory, Phys. Rev. Lett. 82, 5381 (1999)]. In particular, the 2 spin system in 2,3-dibromothiophene may be controlled by external radiofrequency fields such that its spin appears to evolve under the total Hamiltonian of a different quantum system, a truncated harmonic oscillator or a truncated anharmonic oscillator. Thus, not only a time-independent Hamiltonian, but a time-dependent Hamiltonian, can be simulated. Thus, a general control method is enabled.

According to one approach, a femtosecond laser pulse or pulse sequence from, for instance, a system for femtosecond adaptive spectroscopic techniques for coherent anti-Stokes Raman spectroscopy (FAST-CARS), perturbs the vibrational quantum levels of a glucose molecule. The perturbation, in turn, will affect the Raman signal output. The Raman signal is measured and results from the measurement are fed to a processor to determine whether the signal increases or decreases. A controller adjusts the parameters of the laser (e.g., pulse duration, shape, intensity, etc.) of one or more subsequent perturbing laser pulses. This process is repeated until the optimal pulse sequence is obtained to maximize the Raman signal from the target molecule. The laser pulse sequence itself, which is found by this adaptive feedback scheme, can serve as an identifying fingerprint of the molecule.

The signal from a target molecule, nevertheless, may still not be strong enough to be readily detectable. A digital or analog oscillation modulation of a perturbation on a target molecule can confer the benefits of heterodyne detection wherein, only the target molecule evolves according to a prescribed pattern or code. By carefully choosing a series of optical control pulse sequences, it is possible to encode the observed signal with a specified pattern (by using an appropriate input perturbation sequence) to enhance detection efficiency. Provided the response to the series of optical pulses sequences is understood, a detector may monitor the observed signal to look for the pattern expected in response to the input.

A molecule, in response to a perturbation, will respond by yielding a corresponding molecular quantum state. The molecule can be effectively viewed as a “system function.” The molecule maps a set of perturbing pulses to a corresponding set of molecular quantum states, which may be reflected by an observable, such as its Raman spectrum. The mapping of the optimized optical pulse sequence above, to the targeted maximally coherent vibrational state, is but one of an infinite number of such pairs.

A series of perturbations on a system, such as a glucose molecule, may be used to discover the general response of the system to any input perturbation. The system function governs the response of the molecular system (in generating any molecular state) to an arbitrary input. Except for certain limited cases, calculating the system function of most molecular species is generally hard to do analytically. Accordingly, the system function must be usually found experimentally by an iterative (loop) method.

In one embodiment, the system function may be empirically determined by mapping the effects of different input laser pulse sequences to the corresponding Raman responses of a system. Laser pulses may be thought of as an interaction Hamiltonian that can be applied to a molecular system, such as glucose. The interaction Hamiltonian combined with the natural Hamiltonian of glucose, for instance, will govern the molecule's quantum state evolution. The set of possible operators (“quantum computer gates”) available for “computation” are the various laser pulse sequences and the natural Hamiltonian. If the set of operators are diverse enough, then any trajectory in state space of a system may be attained, i.e., any quantum simulation can be performed, so as to arrive at a system function that can characterize the response of the system to a perturbation.

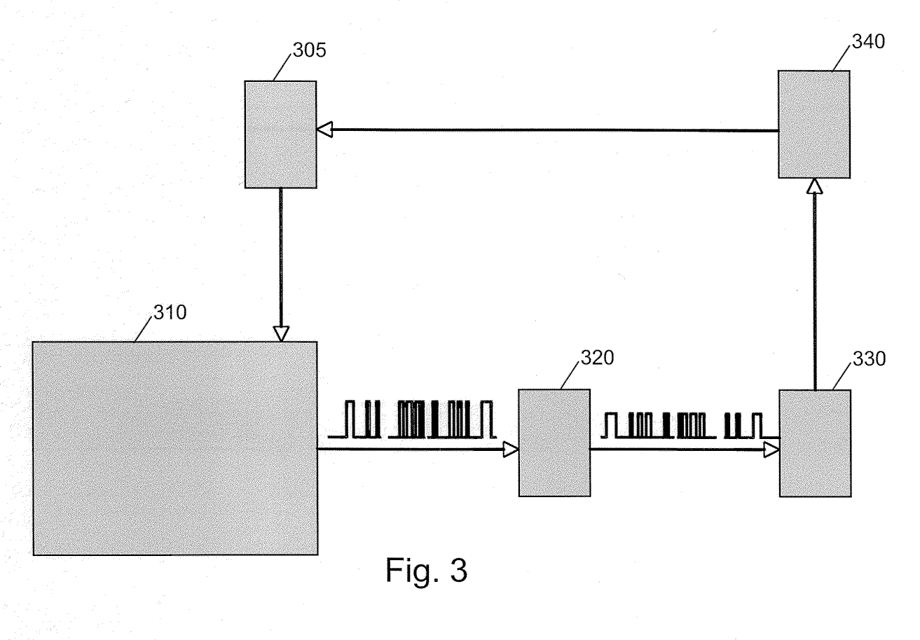

As shown in FIG. 3, to characterize the system function of a system with a feedback loop, external perturbations 310 are applied to a target sample 320, and the responses of target sample 320 are measured by detector 330. A damped harmonic oscillator may be characterized by finding its response to either an impulse perturbation, a swept frequency perturbation, or stochastic input drive perturbations. A pseudorandom binary perturbation sequence, it turns out, is the most effective determinant of a linear oscillator system function. For a nonlinear quantum system, such as glucose, the optimal input needs to be empirically determined, though the Raman modes can be roughly modeled as a damped anharmonic oscillator. If the input (perturbation) set is diverse enough, iterative adaptive control of the perturbation should reveal the system function of the target. Based on the detected response of target sample 320, processor 340 calculates the system function of the target sample 320. From these reiterative measurements, processor 340 calculates the next interactive Hamiltonian to apply to target sample 320, and makes a best guess estimate of the system function. General strategies for achieving each of these steps can be found, for example, in the work by Lennart Ljung cited above. Specific examples relating to optical systems can be found in Judson and in Dudovich also cited above.

To characterize the system function of glucose, a benchmark set of Raman spectra of aqueous glucose is established. This set is obtained through conventional Raman experiments in vitro. Initially, femtosecond laser pulses are used to establish quantum coherence in the glucose vibrational levels, meaning that there is a quantum synchronization of the vibrational states attained by the irradiated population of glucose molecules. The Raman spectra of glucose is monitored, and the preparation and Stokes pulses are adjusted using adaptive control methods in a closed loop adaptive algorithm to explore the system function of the glucose molecule. Once the glucose system function is known or sufficiently characterized, an article of interest, such as an ex vivo biological sample, or in vivo tissue in animal may be examined to determine the amount and/or presence of glucose using a patterned sequence of laser pulses, which is based on the adaptively optimized methods previously used. Based on the calculated system function, we can predict and monitor the expected signal pattern in the Raman spectra for glucose. This modulation and demodulation can also be viewed as an encoding/decoding process. Rather than focusing on a single target state, we look at the pattern of signals resulting from the sequence of applied perturbations. As shown by the high discrimination of a cell-phone signal against a large background, it is possible, through the encoded modulation of the carrier, to make substantial gains in signal to noise on several orders of magnitude.

Preferably, there should be enough time evolution between the perturbation and the detection to make use of the system's complexity. Greater gains in discrimination should be possible if more evolution occurs in the system between the input perturbation and resulting output response. Judicious choices of trajectories in a target molecule's quantum state space, representing the evolution of molecular states under optimal or sub-optimal quantum control pulse sequences, can be used to encode a signal pattern that can assist in extracting the target molecule's signal from a large noisy background.

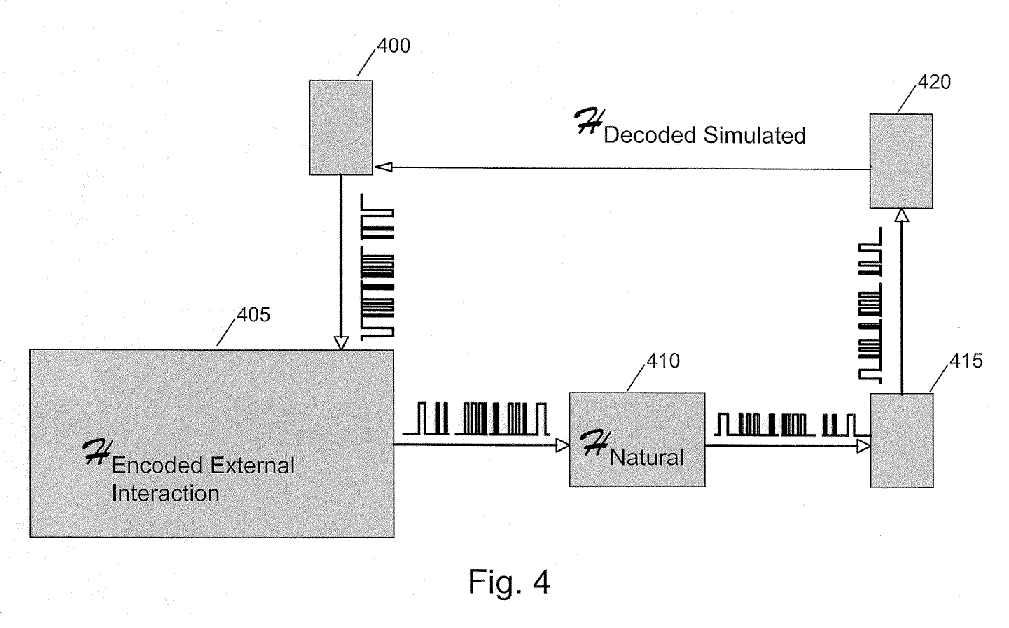

FIG. 4 shows a schematic view of one embodiment for encoding and decoding a pattern onto a target molecule to assist with detection of a target molecule when in the presence of a noisy or large background. A controller 400 equipped with an encoder directs a series of laser pulse sequences, which serve as an encoded interaction Hamiltonian 405, to a target molecule 410 having a natural Hamiltonian. The response of the target 410 is measured by detector 415. To measure the presence of the target molecule, data analyzer 420 compares the measured response to the simulated system based on the known system function of the target molecule and the modulation encoded upon the target 410. In one embodiment, stochastic modulation may be used to significantly enhance the signal-to-noise ratio while reducing other sources of noise, such as background fluorescence.

An additional benefit of using the system identification technique is that not only will the signal-to-noise ratio in the glucose measurement increase but also a continuous representation of the insulin to glucose dynamics, which serves as a real-time diagnostic of the health status of the pancreatic system, can be determined. This approach will also improve on the segmentation of the patient population in terms of diabetic disposition.

The invention having been fully described, it will be apparent to one of ordinary skill in the art that many modifications and changes may be made to it without departing from the spirit and scope of the present invention.

What is claimed is:

1. A quantum control method comprising:
exposing a sample containing a target having a quantum state to a first electromagnetic radiation to enforce a first Hamiltonian on said quantum state of said target, wherein said radiation encodes a pattern on said quantum state of said target;
detecting a response from said sample;
calculating an expected response of said target to said first electromagnetic radiation; and
comparing said expected response of said target to said detected response from said sample to measure the presence of said target in said sample.

2. The quantum control method of claim 1, further comprising:
finding an optimal pulse sequence with a feedback loop to maximize a signal response from said target, wherein the feedback loop comprises:
detecting said signal response from said target after exposing said target to a second electromagnetic radiation to enforce a second Hamiltonian on said quantum state of said target;
calculating a cost function;
modifying said second electromagnetic radiation based on said cost function; and
exposing said target to said modified second electromagnetic radiation.

3. The quantum control method of claim 2, wherein said second electromagnetic radiation is modified according to an adaptive algorithm.

4. The quantum control method of claim 3, wherein said adaptive algorithm is a genetic, landscape algorithm, an algorithm based on evolution strategies, evolutionary programming, simulated annealing, Gaussian adaptation, hill climbing, signal synthesis method based on the system function, or swarm intelligence.

5. The method of claim 1, wherein said first electromagnetic radiation is encoded with a pseudo random binary perturbation pattern, a swept frequency perturbation pattern, a stochastic input drive perturbation pattern, a regular frequency or multiple frequency pattern, or other non-regular perturbation pattern.

6. The method of claim 1, wherein said first electromagnetic radiation is from a laser, NMR, microwave, or radiofrequency generator.

7. The method of claim 6, wherein said first electromagnetic radiation photoacoustically excites said target.

8. The method of claim 6, wherein said response is Raman scattering.

9. The method claim 1, wherein said first Hamiltonian is time dependent or time independent.

10. The method of claim 1, wherein said second Hamiltonian is time dependent or time independent.

11. A detection method comprising:
generating an encoded sequence of perturbations
perturbing a quantum system of a target material in a sample with said encoded sequence of perturbations;
measuring a first signal from said quantum system in response to said encoded sequence of perturbations and using system identification to estimate a system function of said quantum system;
generating a stochastic sequence of perturbations;
perturbing said quantum system of said target material in said sample with said stochastic sequence of perturbations;
measuring a second signal from said quantum system in response to said stochastic sequence of perturbations
decoding said second signal from said quantum system using system identification; and
identifying said target material in said sample based on its system function.

12. The method of claim 10, wherein said stochastic sequence of perturbations comprises a pulse train of electromagnetic radiation having an amplitude and frequency, and wherein at least said amplitude or said frequency of said pulse train is varied.

13. The method of claim 12, wherein said quantum system of said target material is a biological molecule or a therapeutic composition.

14. The method of claim 13, wherein said biological molecule is at least one member selected from the group consisting of glucose, insulin, viral marker, immune marker, cardiovascular biomarker, inflammation biomarker, cholesterol, triglycerides, C-reactive protein, bilirubin, alkaline phosphatase, alanine aminotransferase, AST/GOT, TSH, creatine, creatinine, albumin, cerebral spinal fluid analyte, Tau protein, Alzheimer's biomarker, blood brain barrier transport biomarker, ocular aqueous humor analyte, plaque precursor, cancer antigen, toxicity biomarker, metabolic biomarker, transport biomarker, hemoglobin, diabetes biomarker, central nervous system biomarker, and urogenital biomarker.
